(19) United States
(12) Patent Application Publication
(10) Pub. No.: US 2021/0060282 A1
(43) Pub. Date: Mar. 4, 2021

(54) PORTABLE GAS SUPPLY DEVICE

(71) Applicant: Takashi TAKEHARA, Osaka-shi, Osaka (JP)

(72) Inventor: Takashi TAKEHARA, Osaka-shi, Osaka (JP)

(21) Appl. No.: 16/959,353

(22) PCT Filed: Jan. 8, 2019
(86) PCT No.: PCT/JP2019/000254

§ 371 Date: Jun. 30, 2020

(30) Foreign Application Priority Data
Jan. 9, 2018 (JP) 2018-001531

(51) Int. Cl. A61M 16/12; C25B 15/08; C25B 1/04; C25B 13/08

(52) U.S. Cl. CPC A61M 16/12; C25B 15/08; C25B 1/04; A61M 2202/0208; A61M 2205/8206; A61M 2205/50; A61M 2202/02; C25B 13/08

(57) ABSTRACT

Proposed is a portable gas supply device using electrolysis, capable of controlling emission amount of a desired gas without causing leakage of an electrolytic solution in an electrolysis tank. The portable gas supply device includes: a battery; a control board for controlling power supply from the battery; a pair of positive/negative electrodes to which power from the battery is supplied or blocked by the control board; an electrolysis tank capable of storing water, into which the pair of positive/negative electrodes are inserted; a permeation device through which only a predetermined gas inside the electrolysis tank can permeate; and a nozzle capable of supplying a gas emitted from the permeation device.

### TEHNICAL FIELD

The present invention relates to a portable gas supply device capable of supplying only a desired amount of a gas such as hydrogen gas using an electrolysis method and also capable of preventing water leakage from an electrolysis tank with a simple structure.

### BACKGROUND ART

Recently, effectiveness of hydrogen has been shown in various animal disease experiments such as neurodegenerative diseases and acute lung disorders and human clinical tests for metabolic syndrome, diabetes and the like, and various studies have been actively conducted in medical applications. Intake of hydrogen into the body is recommended particularly for preventing aging or for promoting beauty/health in various states during physical exercises, eating and drinking, suction, stay under ultraviolet/contaminated environments, and under a high stress such as lack of sleep and long-hour work, in which active oxygen tends to be generated easily in the body.

Here, as a conventional hydrogen generation method, there is a method of electrolyzing water, though the method is only a method for generating hydrogen water. The method uses an electrolysis tank having an electrolysis plate disposed therein, and the electrolysis plate has: an ion exchange membrane; a pair of electrode plates closely attached to both sides of the ion exchange membrane; and a fixing part that closely attaches a pair of electrode plates to both sides of the ion exchange membrane. The method includes: filling the electrolysis tank with water; energizing the electrolysis plate to generate hydrogen gas or oxygen gas from the pair of electrode plates; and supplying hydrogen gas and/or oxygen gas through a permeable membrane which is provided in a gas emission hole in an upper portion of the electrolysis tank and allows only gas to permeate therethrough (see, for example, Patent Literature 2). The applicant provides a portable gas supply device with a built-in rechargeable battery that utilizes this electrolysis-type hydrogen generation method and is small and inexpensive so that the user can carry it around freely.

However, in the case of conventional portable gas supply devices, when an electrolytic solution is electrolyzed in an electrolysis tank, the viscosity of the electrolytic solution causes the bubble-like electrolytic solution to rise into air layer in an upper portion of the electrolysis tank. In many cases, the electrolysis tank is filled with the electrolytic solution up to the upper wall. This phenomenon is remarkable as the viscosity of the electrolytic solution increases as the electrolysis progresses. Such state causes a case in which the electrolytic solution reaches the gas permeable membrane in the gas emission hole in the upper portion of the electrolysis tank and the electrolytic solution leaks out. On the other hand, in order to prevent the electrolytic solution from leaking out from the permeable membrane, it is conceivable to employ a permeable membrane made of a material having a small pore diameter of permeable pore.

However, if a material with a too small permeable pore is employed, the permeation rate of hydrogen gas or the like goes down, making it difficult to control the emission amount of hydrogen gas or the like. Alternatively, the permeable membrane extends due to rise in the gas pressure in the electrolysis tank to increase the pore diameter of the permeable pore instead, causing leakage of the electrolytic solution.

Portable gas supply devices are expected to be used not only for sucking hydrogen gas and the like for health promotion and medical purposes, but also for industrial purposes such as hydrogen inspection of fuel cells. Thus, it is considered that needs to delicately control the supply of a desired amount of hydrogen gas will increase in future.

### CITATION LIST

### Patent Literature

Patent Literature 1: Japanese Patent Laid-Open No. 2004-41949

Patent Literature 2: Japanese Patent Laid-Open No. 2014-019640

### SUMMARY OF INVENTION

### Technical Problem

The present invention is designed with respect to the above circumstances, and it is an object of the present invention to provide a configuration in a portable gas supply device using electrolysis, capable of controlling emission amount of a desired gas without causing leakage of an electrolytic solution in an electrolysis tank.

### Solution to Problem

In order to solve the above problems, the present invention provides a portable gas supply device including: a battery; a control board for controlling power supply from the battery; a pair of positive/negative electrodes to which power from the battery is supplied or blocked by the control board; an electrolysis tank capable of storing water, into which the pair of positive/negative electrodes are inserted; a permeation device through which only a predetermined gas inside the electrolysis tank can permeate; and a nozzle capable of supplying a gas emitted from the permeation device. The permeation device has a first permeable membrane and a second permeable membrane in order from an upstream with the electrolysis tank side as the upstream. The first permeable membrane blocks an opening of the electrolysis tank and allows only a predetermined gas to permeate through the first permeable membrane.

The second permeable membrane is disposed so as to be spaced a predetermined distance apart from the first permeable membrane and allows only the gas that has permeated through the first permeable membrane to permeate through the second permeable membrane.

According to the present invention, a gas supply device is provided in which there are provided two permeable membranes that allow only gases such as hydrogen gas or oxygen gas emitted from an electrolysis tank to permeate therethrough, and the gas is emitted to the outside through a two-step permeation process. Another feature is that the first permeable membrane and the second permeable membrane are arranged to be spaced a predetermined distance apart from each other. With this configuration employed, even in a case where emission of hydrogen or the like due to electrolysis causes water to bubble in the electrolysis tank to rise to the upper portion of the electrolysis tank and permeates through the first permeable membrane, the water can be prevented from permeating through the second permeable membrane. This is because there is a distance to the second permeable membrane.

Further, according to this configuration, it is possible to increase the pore diameter of the permeable pores of the permeable membrane employed, compared with in a case where the water permeation is blocked only by the first permeable membrane. As a result, smooth gas emission can be achieved, and the permeable membrane can be easily selected and inexpensive. Further, as compared with in the case where only the first permeable membrane is arranged, it is possible to avoid destabilization of the gas emission amount due to variation of the pore diameter of the first permeable membrane caused by pressure increase and decrease in the electrolysis tank. This also has an advantage of facilitating synchronization of emission control of a desired gas amount with electrical control. This is also advantageous from the viewpoint of preventing the water with a change in quality by electrolysis from emission, using a simple configuration.

Note that the first permeable membrane is preferably a fluororesin porous film having selective permeability.

Further, it is preferable that the permeation device is mounted onto an opening in an upper portion of the electrolysis tank, the first permeable membrane blocks the inside of the electrolysis tank from the inside of the permeation device, and the second permeable membrane blocks the inside of the permeation device from the outside of the permeation device.

Specifically, the permeation device in the gas supply device is configured such that the permeation device is provided with the first permeable membrane and the second permeable membrane and can be mounted onto an opening in an upper portion of the electrolysis tank.

Further, the permeation device may be provided with a liquid pool portion, which stores the liquid having leaked from the first permeable membrane, in a space from the first permeable membrane to the second permeable membrane. The permeation device, even if water leaks from the first permeable membrane, can flow the water into the liquid pool portion at the side to store and drain the water.

More specifically, the permeation device includes: a lid member having an opening at its upper portion and mounted onto an upper portion of the electrolysis tank; blocking members mounted onto an upper portion of the lid member, which block the communication with an opening of the lid member by the first permeable membrane, and block the communication with the outside above by the second permeable membrane; a liquid pool portion for storing liquid, which has leaked from the first permeable membrane into a space from the first permeable membrane to the second permeable membrane, by flowing the liquid in a laterally downward direction of the first permeable membrane; and a drain hole for discharging the liquid stored in the liquid pool portion to the outside.

### Advantageous Effects of Invention

According to the present invention, in the portable gas supply device using electrolysis, two permeable membranes are disposed with a space therebetween. Thereby, when hydrogen gas or the like is emitted from the electrolysis tank, only a desired amount of hydrogen gas or the like can be emitted without leaking the electrolytic solution to the outside. Moreover, when the permeation device of the portable gas supply device is used, the device does not aim at preventing leakage of the electrolytic solution at once but tolerates some leakage at the first step, and aims at complete leakage prevention at the second step. Thus, it is possible to prevent the internal pressure from rising in the electrolysis tank to stabilize the emission amount of gas.

### BRIEF DESCRIPTION OF DRAWINGS

FIG. 1 shows a block diagram schematically representing an embodiment of a portable gas supply device of the present invention.

FIG. 2 shows views of the portable gas supply device of the present invention as seen from individual directions, in which FIG. 2(a) is a left side view, FIG. 2(b) is a front view, FIG. 2(c) is a right side view, FIG. 2(d) is a top view, and FIG. 2(e) is a sectional views in the front view direction.

FIG. 3 is an assembling/disassembling view of individual members of an electrolysis tank and parts around the tank of the portable gas supply device of the present invention.

FIG. 4 is a perspective view of an electrolysis tank and parts around the tank of the portable gas supply device of FIG. 3.

### DESCRIPTION OF EMBODIMENTS

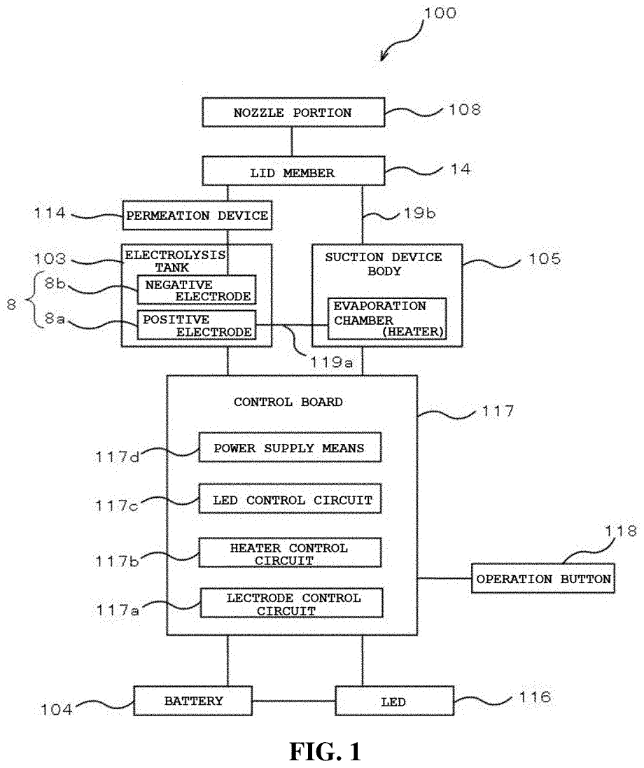

Representative embodiments of the portable gas supply device of the present invention are described below in detail with reference to FIGS. 1 to 4, but it goes without saying that the present invention is not limited to the illustrated ones. Moreover, since each drawing is for conceptually describing the present invention, dimensions, ratios or numbers may be exaggerated or simplified for easy understanding as necessary. Furthermore, in the following description, the same or corresponding parts are denoted by the same reference numerals and/or reference characters, and redundant description may be omitted.

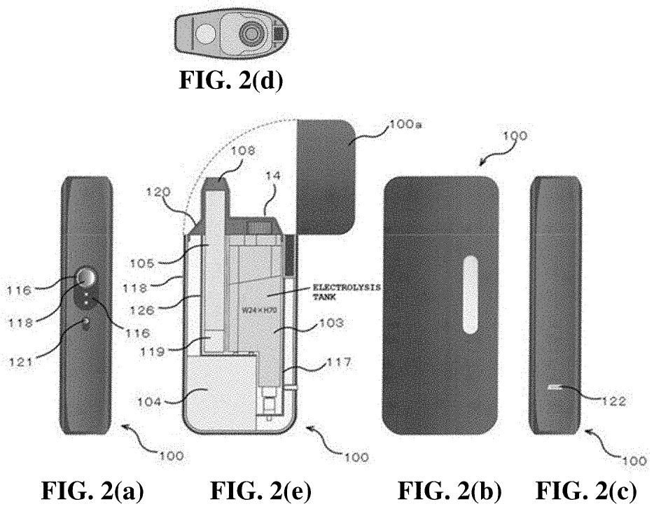

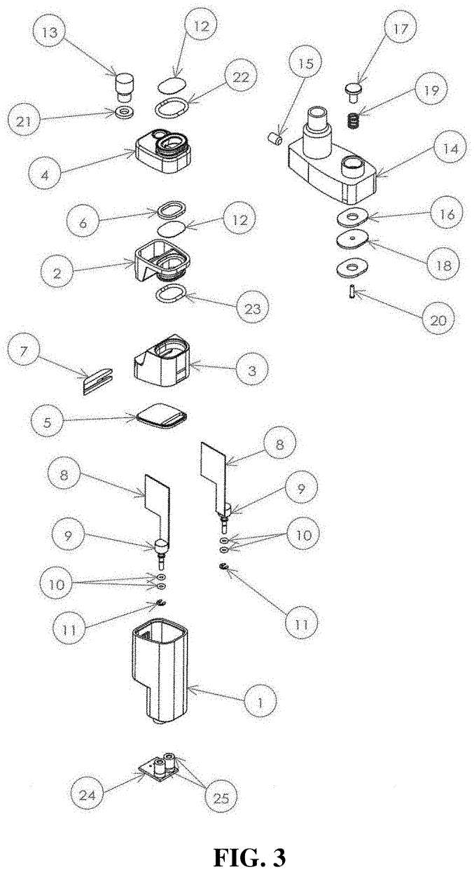

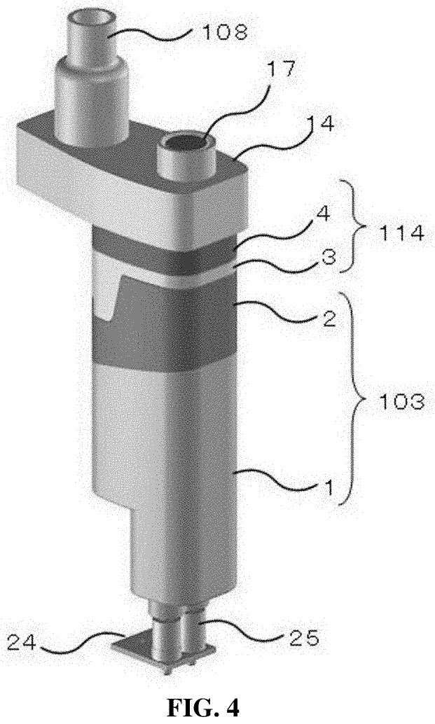

FIG. 1 is a block diagram schematically showing the portable gas supply device 100. Further, FIG. 2 shows views of the portable gas supply device 100 of the present invention as seen from individual directions, in which FIG. 2(a) is a left side view, FIG. 2(b) is a front view, FIG. 2(c) is a right side view, FIG. 2(d) is a top view, and FIG. 2(e) is a sectional view in the front view direction. An up-and-down direction and a vertical direction herein mean the up-and-down direction and the vertical direction on the paper of FIG. 2(b), respectively. A width direction, a horizontal direction, and a lateral direction herein mean the left-and-right direction, the horizontal direction and the left-and-right lateral direction on the paper of FIG. 2(b), respectively. Further, FIG. 3 shows an assembling/disassembling view exemplifying individual members of an electrolysis tank 103 and parts around the tank of the portable gas supply device 100, and FIG. 4 shows a perspective view of the electrolysis tank 103 and the parts around the tank of the portable gas supply device 100 of FIG. 3.

The portable gas supply device 100 is outlined below with reference to FIGS. 1 and 2, and the electrolysis tank 103 and parts around the tank of the portable gas supply device 100 are described below with reference to FIGS. 3 to 4.

As shown in FIG. 1, the portable gas supply device 100 is generally configured with a battery 104, an LED 116, control means 117, an electrolysis tank 103, a suction device body 105, a lid member 14, and a nozzle portion 108. First, the battery 104 is a rechargeable battery such as a lithium ion battery, and a pair of positive/negative electrodes 8a and 8b are disposed in the electrolysis tank 103. The positive/negative electrodes 8a and 8b are supplied with electric power from the battery 104 via the control means (control board) 117, and the LED 116 is connected to the battery 104. The control board 117 includes an electrode control circuit 117a, a heater control circuit 117b, an LED control circuit 117c, and power supply means (power supply circuit) 117d.

FIGS. 1 and 2 show, as an example of the portable gas supply device 100, an example in which the suction device body 105 is inserted and disposed in addition to supply of hydrogen gas and oxygen gas. However, in the portable gas supply device 100 of the present invention, an aromatic supply device may be disposed in addition to, or instead of the suction device body 105. Also, in a case of industrial use, only the hydrogen gas and oxygen gas supply device may be disposed. In the example of FIGS. 1 and 2, a pressure sensor switch 119 is provided at the bottom portion of a receiving portion of the suction device body 105. When the lower end of the suction device body 105 presses the pressure sensor switch 119, the power supply means 117d of the control board 117 issues a power supply command so that the power of the battery 104 is ready to be supplied to the suction device body 105.

When the user operates the operation means (operation button) 118 by a finger, the electrode control circuit 117a controls energization and interruption of the pair of electrodes 8a, 8b in the electrolysis tank 103 in response to the operation, and the power supply means 117d varies the amount of power supplied from the battery 104 to supply power to the electrodes 8a and 8b. When power is supplied to the pair of electrodes 8a and 8b, the electrolytic solution (for example, sodium citrate aqueous solution) stored in the electrolysis tank 103 is electrolyzed. As a result, oxygen is generated on the positive electrode 8a side and hydrogen is generated on the negative electrode 8b side.

Hydrogen generated from the negative electrode 8b flows into the lid member 14 via a permeation device 114 mounted onto an upper portion of the electrolysis tank 103. Further, oxygen generated from the positive electrode 8a may be vented when flowing into the lid member 14 as described below.

Further, in the suction device body 105, when the pressure sensor switch 119 is turned on, the power supply means 117d supplies electric power from the battery 104 to the heater in the suction device body 105 to heat a suction cartridge attached to an internal vapor chamber (not shown). When the suction cartridge is heated by the heater, vapor containing nicotine or the like is generated. Note that the suction cartridge is a disposable replacement of a heating type electronic suction device containing drugs, fragrances, etc., and generates a nicotine-containing vapor by heating. Other cartridges include those that generate aromatic vapor containing nicotine or the like by heating, or those that contain aromatic without containing nicotine to generate aromatic vapor by heating.

The vapor containing nicotine or the like generated in the suction device body 105 is emitted into the mouth by sucking the nozzle portion 108. At this time, the negative pressure generated by sucking causes the hydrogen emitted from the permeation device 114 to flow in the lid member 14. The hydrogen passes through a gap between the periphery of an upper portion of the suction device body 105 exposed in the lid member 14 and the inner wall of the nozzle portion 108. The hydrogen is mixed with nicotine-containing air therein and guided into the mouth or emitted outside. It is also conceivable to guide only hydrogen into the mouth or to the outside without heating the suction device body 105.

FIG. 2 shows a specific configuration example of the portable gas supply device 100 with the suction device body 105 inserted. In FIGS. 2(a), 2(b), and 2(c), which are a left side view, a front view, and a right side view, respectively, an open/close lid 100a of the portable gas supply device 100 is closed. In FIGS. 2(d) and 2(e), which are a plan view and a sectional view, respectively, the opening/closing lid 100a of the portable gas supply device 100 is removed. The portable gas supply device 100 has a tubular suction device receiving portion (hereinafter, also referred to as a “receiving portion”) 120 extending downward from an opening on the upper left side, with the opening/closing lid 100a removed (open). The receiving portion 120 has the suction device body 105 inserted thereinto. The suction device body 105 is a body unit of a general-purpose cylindrical heating type electronic suction device.

The bottom portion of the receiving portion 120 of the portable gas supply device 100 has the pressure sensor switch 119 disposed thereon. When the pressure sensor switch 119 is pressed, electric power is supplied from a rechargeable battery (a lithium battery) 104 to heat the suction cartridge in the suction device body 105 so that the vapor containing nicotine or the like is ready to be sucked. In the portable gas supply device 100, the rechargeable battery 104 functions as a substitute for batteries in general-purpose cylindrical heating type electronic suction devices.

Furthermore, the portable gas supply device 100 has an operation button (main power source/hydrogen button) 118, an LED indicator 116, and an electronic suction device ON/OFF switch 121 provided on the left side (see FIG. 2(e)). The electronic suction device ON/OFF switch 121 is an ON/OFF switch of the pressure sensor switch 119. When the electronic suction device ON/OFF switch 121 is ON, power of the rechargeable battery 104 is ready to be supplied to the suction device body 105. When the electronic suction device ON/OFF switch 121 is OFF, power is not supplied from the rechargeable battery 104 even if the pressure sensor switch 119 is pressed. The main power source/hydrogen button 118 is a button type power supply switch for the positive/negative electrodes 8 in the electrolysis tank 103 and the main power source to be described below, and serves as both ON/OFF of the main power source and ON/OFF of power supply to the positive/negative electrodes 8 depending on the pressing way and time.

In this example, first, when a charger (USB cable (not shown)) is connected to the charging terminal 122, three LEDs 116, 118 of red, yellow, and green, one of which is located around the main power source/hydrogen button 118, sequentially blink once at a predetermined frequency, and the corresponding two LEDs 116 at the bottom and middle blink twice in accordance with the battery level. Repeating five-time pressing of the main power source/hydrogen button 118 three times in a row turns on the power, and a five-time pressing turns off the LED 116 that has been on according to the battery level and turns off the power source.

When the power source is turned on, the mode goes into a mode of suction device body 105 and hydrogen generation (normal mode). When the LEDs 116 and 118 comes on in blue for electrolysis confirmation and the main power source/hydrogen button 118 is pressed, the suction device body 105, and electrolysis caused by energizing the positive/negative electrodes 8 operate at the same time. The operation stops at the same time when the finger is released from the main power source/hydrogen button 118. (In this mode, the operation of energizing/heating the suction device body 105 is controlled to be delayed by 1 second from the operation of energizing/electrolyzing the positive/negative electrodes 8.) Pressing the switch button three times in a row with the mode of heating type suction device and hydrogen generation (normal mode) shifts the mode to a mode of hydrogen-only, and the electrolysis confirmation LED (blue) blinks as breathing (slowly blinks), and only electrolysis operates.

When the main power source/hydrogen button 118 is pressed with the mode of suction device body 105 and hydrogen generation (normal mode), one of the three LEDs 116, 118 (red, yellow, green) around the main power source/hydrogen button 118 comes on, depending on the battery level, and power supply to a coil starts. When the finger is released from the main power source/hydrogen button 118, the LEDs 116 and 118 turn off and the power supply to the suction device body 105 is stopped. Here, when the electrolysis tank 103 is filled with the electrolytic solution, energization to the positive/negative electrodes 8 and electrolysis are operated at the same time while the main power source/hydrogen button 118 is pressed. Further, with the power on, regardless of the operation mode, when the main power source/hydrogen button 118 is pressed, energization to the positive/negative electrodes 8 and electrolysis is started, and when the finger is released from the main power source/hydrogen button 118, energization to the positive/negative electrodes 8 and electrolysis operation is stopped. Here, the lighting of each of the LEDs 116 and 118 is controlled by an internal indicator board 126.

Next, with reference to FIGS. 3 to 4, the inside of the electrolysis tank 103, the permeation device 114 mounted thereon, and the like are described below. As shown in FIGS. 3 to 4, the electrolysis tank 103 is configured with an electrolysis tank body 1 and an electrolysis tank lid portion 3 (the electrolysis tank lid portion 3 also functions as a part of the permeation device). The electrolysis tank body 1 is a container for storing the electrolytic solution that extends in the up-and-down direction, has a shape in which the lower portion has a smaller thickness than the upper portion, and is an integrally formed body that is fluidically connected to each other inside. The electrolysis tank body 1 can be filled with water from an upper opening, and has a plate-shaped separator 5 having a through hole inserted and an electrolysis tank lid portion 3 mounted, at an upper portion of the opening, to be closed. The electrolysis tank lid portion 3 is a case that penetrates vertically, and has a two-step shape in which the lower portion has a larger thickness than the upper portion. The lower portion of the electrolysis tank lid portion 3 is fixed to the separator 5 by a lock lever 7 to form a bottom portion. Further, the opening in an upper portion of the electrolysis tank lid portion 3 has a spot facing shape formed to receive a first permeable member 2 of the permeation device to be described below.

Further, in the electrolysis tank body 1, the lower portion has a smaller thickness than the upper portion. So, even if the aqueous solution stored inside is electrolyzed and the amount of stored water is reduced, the electrolytic solution is still stored to the extent that most portion of the pair of positive/negative electrodes 8 are immersed in the electrolytic solution. As a result, the air layer in the upper portion of the electrolysis tank body 1 is reduced and the electrolysis performance is ensured. On the other hand, when the liquid level of the electrolytic solution rises to the limit and the viscosity increases due to electrolysis, the bubbles generated by electrolysis penetrate into and stay in the air layer or the electrolysis tank lid portion 3 even if there is a separator 5.

Two positive/negative electrodes (mesh electrodes) 8 form a pair, are directed upward, and are arranged in parallel in the longitudinal direction. The electrodes form an anode and a cathode, respectively, and are supplied with power from the battery 104. Further, the positive/negative electrodes 8 have an upper portion larger than a lower portion so as to correspond to the portion with a smaller thickness and the portion with a larger thickness of the electrolysis tank body 1. The positive/negative electrodes 8 has a rod-shaped titanium electrode 9 connected to the lower end thereof, so that the positive/negative electrode 8 can stand upright on a terminal board 24 and are electrically connected thereto. In order that the positive/negative electrodes 8 and the terminal board 24 are shielded from water with the positive/negative electrodes 8 standing upright, there are provided sockets 25 (made of a resin such as silicone) attached to the terminal board 24 and O-rings 10 and 11 (made of a resin such as silicone; hereinafter, the same applies to the O-ring) attached around the titanium electrode 9.

The upper portion of the electrolysis tank lid portion 3 has the permeation device attached thereto. First, the upper portion of the electrolysis tank lid portion 3 has a first permeable member 2 mounted thereto. The first permeable member 2 has a lower portion having a smaller thickness and protruding downward so as to fit vertically with the electrolysis tank lid portion 3, and an upper portion largely opened upward. The portion with a smaller thickness of the first permeable member 2 is closed at a bottom portion, connected to the opening at the upper portion, and formed so as to be a liquid pool. Further, the portion with a larger thickness in the upper portion of the first permeable member 2 is connected to the opening of the liquid pool on the side of the portion with a smaller thickness described above, and has a through hole fluidically connected to the opening of the electrolysis tank lid portion 3. The lower end of the through hole is inserted into and connected to the opening of the electrolysis tank lid portion 3 by using the opening as a spot facing. At this time, the through hole of the first permeable member 2 and the opening of the electrolysis tank lid portion 3 have an O-ring 23 disposed therebetween for preventing water leakage.

Further, the through hole of the first permeable member 2 has a first permeable membrane 12 disposed therein by a permeable membrane retainer 6 to close the through hole. The first permeable membrane 12 is a resin porous membrane having a selective permeability that allows gas to permeate therethrough and blocks liquid while adjusting the internal pressure with micropores. Here, a tetrafluoroethylene resin porous membrane (“TEMISH” manufactured by Nitto Denko Corporation) is used (the same applies to the second permeable membrane 12 described below). As a first step, the first permeable membrane 12 blocks bubbles of the electrolytic solution that have reached the inside of the electrolysis tank lid portion 3. However, a rise of the internal pressure inside the electrolysis tank body 1 may cause the first permeable membrane 12 to extend, which expands the micropores to allow bubble-like electrolytic solution to permeate through the membrane. The rise of the internal pressure may also cause gasified electrolytic solution to permeate through the membrane and penetrate into the first permeable member 2. On the other hand, it is also undesirable to make the pore diameter of the first permeable membrane 12 too small to reduce the hydrogen permeation rate. Therefore, the first permeable member 2 tolerates the electrolytic solution penetrating thereinto to a certain degree, and has its portion with a smaller thickness described above to store the electrolytic solution using as a liquid pool.

Further, the first permeable member 2 has a second permeable member 4 mounted on the upper portion. The second permeable member 4 has a downward opening (though not shown), and matches the upper opening of the first permeable member 2 to configure an internal space. The upper portion of the second permeable member 4 has a through hole formed at a position where the through hole of the electrolysis tank lid portion 3 and the through hole of the first permeable member 2, both described above, are visible. The through hole is closed by a second permeable membrane 12 and sealed by an O-ring 22, as is the same as in the case of the permeable membrane (first permeable membrane 12) of the first permeable member 2. The second permeable membrane 12 is also a resin porous membrane having a selective permeability that allows gas to permeate therethrough and blocks liquid, and a tetrafluoroethylene resin porous membrane is used here.

In the first step described above, the penetration of the electrolytic solution from the electrolysis tank is almost blocked, but in the second step, the second permeable membrane 12 prevents the electrolytic solution from being further emitted to the outside. In the first permeable membrane as the first step, smooth permeation of gas is prioritized over complete blocking of the electrolytic solution. So, the internal pressure in the space between the first permeable member 2 and the second permeable member 4 does not rise. Thus, the selective porous resin membranes of the same quality allow smooth permeation of hydrogen gas and the like, while achieving further blocking of the electrolytic solution. Furthermore, the second permeable member 4 is provided with a hole for draining the electrolytic solution stored in the liquid pool of the first permeable member 2, and the hole is closed by a screw 13 via a packing 21. At the time of draining, the screw 13 is removed so that the electrolytic solution can be disposed.

The upper portion of the second permeable member 4 has the lid member 14 mounted thereto from above. The upper portion of the lid member 14 is provided with the nozzle portion 108 for sucking as well as a through hole above the second permeable membrane 12, into which a valve shaft 17 is inserted to close the hole. An end of the valve shaft 17 is connected by a pin 20 to a base 18 sandwiched by packings 16. Thus, the valve shaft normally opens the through hole by the action of a spring 19 and closes the through hole when a negative pressure acts on the inside of the lid member 14 by sucking the nozzle portion 108. This is made so that, during sucking, the hydrogen gas and the like is concentrated in the direction of the nozzle portion 108 by closing, and during non-sucking, the hydrogen gas and the like is not overfilled to avoid an excessively high internal pressure.

As shown in FIG. 2, when the nozzle portion 108 of the lid member 14 is sucked, hydrogen gas passes through the electrolysis tank body 1, electrolysis tank lid portion 3, the first permeable member 2, and the second permeable member 4 in this order, flows inside, and reaches the nozzle portion 108. The hydrogen passes through a gap between the nozzle portion 108 and the upper end of the suction device body 105, mixes with the gas from the suction device body 105, and is emitted into the mouth of the user or the outside. In the case of the portable gas supply device 100 that does not include the suction device body 105 or does not operate the suction device body 105, hydrogen gas (or oxygen gas) is emitted from the nozzle portion 108 into the mouth of the user or to the outside.

As described above, the portable gas supply device of the present invention, particularly the permeation device for hydrogen gas and the like from the electrolysis tank, has been described by exemplifying the embodiments, but the present invention is not limited to this, and those skilled in the art will understand that other variations and improvements can be obtained within a range without departing from the spirit or teachings of the claims, the description and the like.

### INDUSTRIAL APPLIACBILITY

According to the portable gas supply device of the present invention, disposing two permeable membranes in a certain space in a portable gas supply device using electrolysis allows emission of only a desired amount of hydrogen gas and the like without leakage of the electrolytic solution to the outside during emission of hydrogen gas from the electrolysis tank. Moreover, when the permeation device of the portable gas supply device is used, the device does not aim at preventing leakage of the electrolytic solution at once but tolerates some leakage at the first step, and aims at complete leakage prevention at the second step. Thus, it is possible to prevent the internal pressure from rising in the electrolysis tank to stabilize the emission amount of gas. Therefore, the present invention can be utilized for controlling delicate sucking of hydrogen gas or the like according to physical conditions, or for industrial inspection in which emission amount of hydrogen gas or the like is strictly controlled.

### REFERENCE SIGNS LIST

1 electrolysis tank body

2 first permeable member

3 electrolysis tank lid portion

4 second permeable member

8 positive/negative electrodes

8a positive electrode

8b negative electrode

12 permeable membrane (first permeable membrane, second permeable membrane)

13 screw

14 lid member

17 valve shaft

19 spring

16 packing

18 base

20 pin

21 packing

22 O-ring

100 portable gas supply device

100a open/close lid

103 electrolysis tank

104 battery

105 suction device body

108 nozzle portion

114 permeation device

116 LED (LED indicator)

117 control board (control means)

118 operation button (main power source/hydrogen button)

119 pressure sensor switch

120 suction device receiving portion (receiving portion)

122 charging terminal

126 indicator board

1. A portable gas supply device comprising:
a battery;
a control board for controlling power supply from the battery;
a pair of positive/negative electrodes to which power from the battery is supplied or blocked by the control board;
an electrolysis tank capable of storing water, the electrolysis tank into which the pair of positive/negative electrodes are inserted;
a permeation device through which only a predetermined gas inside the electrolysis tank can permeate; and
a nozzle capable of supplying a gas emitted from the permeation device,
wherein the permeation device has a first permeable membrane and a second permeable membrane in order from an upstream with the electrolysis tank side as the upstream, the first permeable membrane blocking an opening of the electrolysis tank, the first permeable membrane allowing only a predetermined gas to permeate through the first permeable membrane, the second permeable membrane being disposed so as to be spaced a predetermined distance apart from the first permeable membrane, the second permeable membrane allowing only the gas that has permeated through the first permeable membrane to permeate through the second permeable membrane.

2. The portable gas supply device according to claim 1, wherein the first permeable membrane is a fluororesin porous film having selective permeability.

3. The portable gas supply device according to claim 2, wherein
the permeation device is mounted onto an opening in an upper portion of the electrolysis tank, the first permeable membrane blocks the inside of the electrolysis tank from the inside of the permeation device, and the second permeable membrane blocks the inside of the permeation device from the outside of the permeation device.

4. The portable gas supply device according to claim 3, wherein
the permeation device is provided with a liquid pool portion in a space from the first permeable membrane to the second permeable membrane, the liquid pool portion storing liquid leaked from the first permeable membrane.

5. The portable gas supply device according to claim 4, wherein
the permeation device comprises:
a lid member mounted onto an upper portion of the electrolysis tank, the lid member having an opening at an upper portion of the lid member;
blocking members mounted onto an upper portion of the lid member, the blocking members blocking the communication with an opening of the lid member by the first permeable membrane, and the blocking members blocking the communication with the outside above by the second permeable membrane;
a liquid pool portion for storing liquid by flowing the liquid in a laterally downward direction of the first permeable membrane, the liquid having leaked from the first permeable membrane into a space from the first permeable membrane to the second permeable membrane; and
a drain hole for discharging the liquid stored in the liquid pool portion to the outside.
